(19) United States
(12) Patent Application Publication
(10) Pub. No.: US 2019/0209645 A1
(43) Pub. Date: Jul. 11, 2019

(54) POTENTIATION OF ANTIBIOTIC ACTIVITY BY A NOVEL CATIONIC PEPTIDE, SPR741

(71) Applicant: SPERO POTENTIATOR, INC., Cambridge, MA (US)

(72) Inventor: Troy LISTER, Walpole, MA (US)

(21) Appl. No.: 16/301,254

(22) PCT Filed: May 12, 2017
(86) PCT No.: PCT/US2017/032455

§ 371 Date: Nov. 13, 2018

Related U.S. Application Data
(60) Provisional application No. 62/336,177, filed on May 13, 2016.

(51) Int. Cl. A61K 38/12; A61K 31/439; A61K 31/7048; A61K 31/427; A61K 9/00; A61P 31/04

(52) U.S. Cl. CPC A61K 38/12; A61K 31/439; A61K 31/7048; A61P 31/04; A61K 9/0014; A61K 9/0019; A61K 31/427

(57) ABSTRACT

This disclosure provides a method of potentiating the efficacy of certain antibiotics by administering a therapeutically effect amount of the antibiotic in combination with SPR741. In certain embodiments the antibiotic is retapamulin, telithromycin, or aztreonam. The disclosure also provides a method of treating a bacterial infection by administering SPR741 in combination with a second antibiotic and pharmaceutical compositions comprising SPR741 and a second antibiotic.

### CROSS REFERENCE TO RELATED APPLICATION

This application claims priority from U.S. provisional application No. 62/336,177, filed May 13, 2016, which is hereby incorporated by reference in its entirety.

### BACKGROUND

Gram-negative bacteria cause more than 40% of all septicemic infections and many of the Gram-negative bacteria are resistant to multiple antibiotics. Gram-negative bacteria possess lipopolysaccharide as a component of the outer membrane, which inhibits the diffusion of many antibacterial agents deeper into the cell, where their ultimate targets are located. Many antibacterial agents effective against Gram-positive bacteria lack activity against Gram-negative bacteria.

Polymyxins are a group of closely related antibiotic substances produced by strains of Paenibacillus polymyxa and related organisms. These cationic drugs are relatively simple peptides with molecular weights of about 1000. Polymyxins, such as polymyxin B, are decapeptide antibiotics, i.e., they are made of ten (10) aminoacyl residues. They are bactericidal and especially effective against Gram-negative bacteria such as Escherichia coli and other species of Enterobacteriaceae, Pseudomonas, Acinetobacter baumannii, and others. However, polymyxins have severe adverse effects, including nephrotoxicity and neurotoxicity. These drugs thus have limited use as therapeutic agents because of high systemic toxicity.

Polymyxins were widely used in the therapy of serious infections caused by those bacteria since their discovery in the 1950's, but because of the toxicity, their use was largely abandoned in the 1970's when newer, better tolerated antibiotics were developed. The recent emergence of multiresistant strains of Gram-negative bacteria has resulted in many of the less toxic antibiotics losing their effectiveness against Gram negative bacteria. Polymyxins have maintained their effectiveness against these emergent multiresistant strains of Gram-negative bacteria. Accordingly, polymyxins have been recalled to the therapeutic arsenal, although, due to their toxicity, they are considered a therapeutic of last resort. Their systemic (i.e., non-topical) use is, however, largely restricted to the therapy of life-threatening infections caused by multiply resistant strains of Pseudomonas aeruginosa and A. baumannii as well as by carbapenem-resistant Enterobacteriaceae.

SPR741, Pub Chem ID 53323381, has the formula Acetyl-Thr-dSer-cy[Dab-Dab-dPhe-Leu-Dab-Dab-Thr], where Dab is an α,γ-diamino-n-butyryl residue and cy is cyclic, which is also shown below as a chemical structure.

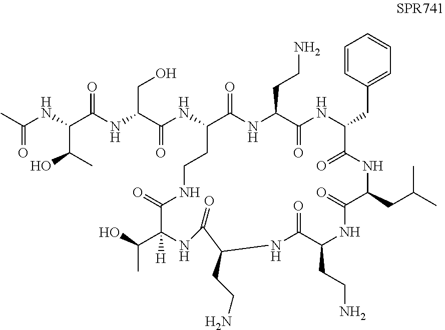 [SMILES: CC(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H](CC(C)C)NC(=O)[C@@H](Cc2ccccc2)NC(=O)[C@H](CCN)NC1=O)[C@@H](C)O]

SPR741 has previously been shown to increase the sensitivity of certain bacteria to Mupirocin, Azithromycin, Fusidic Acid, and Vancomycin. SPR741 permeabilizies the outer membrane of Gram negative bacteria thus granting antibiotics that would otherwise be excluded access to their targets when administered in combination with SPR741.

### SUMMARY

The disclosure provides a method of treating a bacterial infection in a subject comprising administering a therapeutically effective amount of a combination of SPR741 and an antibiotic selected from retapamulin, telithromycin, aztreonam, and combinations thereof to the subject.

A pharmaceutical composition comprising SPR741 and at least one antibiotic selected from retapamulin, telithromycin, and aztreonam.

### DETAILED DESCRIPTION

The in vitro potency of combinations of the cationic peptide, SPR741 with selected antibiotics against MDR and clinical isolates of Escherichia coli (Ec), Klebsiella pneumoniae (Kp), and Acinetobacter baumannii (Ab) has been assessed. SPR741 effectively reduced the MIC of currently utilized antibiotics against MDR isolates of Ab, Ec, and Kp, extending the promising spectrum of activity of these combinations, and supporting continued development of SPR741 for the treatment of Multi Drug Resistant Gram negative bacteria.

Treatment of human patients is particularly contemplated. However, treatment of non-human subjects is within the scope of the disclosure. The disclosure includes treatment or prevention of microbial infections in fish, amphibians, reptiles or birds, but a preferred embodiment of the disclosure includes treating mammals.

A therapeutically effective amount of a pharmaceutical composition/combination is an amount effective, when administered to a subject, to provide a therapeutic benefit, such as to decrease the morbidity and mortality associated with bacterial infection and/or effect a cure. In certain circumstances a subject suffering from a microbial infection may not present symptoms of being infected. Thus a therapeutically effective amount of a compound is also an amount sufficient to significantly reduce the detectable level of microorganism in the subject's blood, serum, other bodily fluids, or tissues. The disclosure also includes, in certain embodiments, using compounds of the disclosure in prophylactic treatment and therapeutic treatment. In the context of prophylactic or preventative treatment, a “therapeutically effective amount” is an amount sufficient to significantly decrease the incidence of or morbidity and mortality associated with bacterial infection. For example, prophylactic treatment may be administered when a subject is known to be at enhanced risk of bacterial infection, such cystic fibrosis or ventilator patients. A significant reduction is any detectable negative change that is statistically significant in a standard parametric test of statistical significance such as Student's T-test, where p<0.05.

“Pharmaceutical compositions” are compositions comprising at least one active agent, such as a SPR741, and at least one other substance, such as an antibiotic, or a carrier. Pharmaceutical compositions meet the U.S. FDA's GMP (good manufacturing practice) standards for human or non-human drugs. The term “carrier” applied to pharmaceutical compositions/combinations of the disclosure refers to a diluent, excipient, or vehicle with which an active compound is provided.

The pharmaceutical compositions of the disclosure include ocular, oral, nasal, transdermal, topical with or without occlusion, intravenous (both bolus and infusion), inhalable, and injection (intraperitoneally, subcutaneously, intramuscularly or parenterally) formulations. The composition may be in a dosage unit such as a tablet, pill, capsule, powder, granule, liposome, sterile ocular solution, parenteral solution or suspension, metered aerosol or liquid spray, drop, ampoule, auto-injector device, or suppository; for administration ocularly, orally, intranasally, sublingually, parenterally, or rectally, or by inhalation or insufflation.

The dosage form containing the composition of the disclosure contains an effective amount of the active agent necessary to provide a therapeutic effect by the chosen route of administration. The composition may contain from about 5,000 mg to about 0.5 mg (preferably, from about 1,000 mg to about 0.5 mg) of a compound of the disclosure or salt form thereof and may be constituted into any form suitable for the selected mode of administration. The dosage form may be formulated for immediate release or controlled release, including delayed release or sustained release. The pharmaceutical composition includes SPR741 and at least one direct acting antibiotic (a compound efficacious for killing pathogenic bacteria in vivo) for example, retapamulin, telithromycin, aztreonam.

### Specific Embodiments

The disclosure includes method of treating a bacterial infection in a subject comprising administering a therapeutically effective amount of a combination of SPR741 and an antibiotic selected from retapamulin, telithromycin, aztreonam, and combinations thereof to the subject.

In certain embodiments the bacterial infection is an E. coli infection, a Klebsiella pneumoniae infection, or an Acinetobacter baumannii infection.

The disclosure includes embodiments in which:

(1) The antibiotic is retapamulin.

(2) The antibiotic is telithromycin.

(3) The antibiotic is aztreonam.

(4) The subject is a mammal.

(5) The subject is a human patient.

(6) The retapamulin is administered as a topical formulation containing SPR741 and less than 1 mg retapamulin per gram formulation.

(7) The telithromycin is administered orally and 10 mg to 300 mg, or 10 mg to 200 mg, or 10 mg to 100 mg, telithromycin are administered daily.

(8) aztreonam is administered intravenously and less than 500 mg, less than 400 mg, less than 250 mg aztreonam, or less than 100 mg are administered per intravenous infusion. In certain embodiment the intravenous infusion is a 30 minute infusion.

The disclosure includes a pharmaceutical composition comprising SPR741 and at least one antibiotic selected from retapamulin, telithromycin, and aztreonam.

The disclosure additionally comprises a pharmaceutically acceptable carrier.

The disclosure includes pharmaceutical compositions in which the antibiotic is retapamulin, the composition is a topical composition and the composition contains less than 1 mg retapamulin per gram formulation.

The disclosure includes pharmaceutical compositions in which the antibiotic is telithromycin, the composition is an oral dosage form formulated for once daily administration and the dosage form contains 10 mg to 300 mg telithromycin.

The disclosure includes pharmaceutical compositions in which the antibiotic is aztreonam, the composition is an injectable or intravenous composition, and the composition contains less than 250 mg aztreonam per injection or infusion.

### EXAMPLES

### Example 1. Assessment of Potency of SPR741 in Combination with Certain Antibiotics

The potency of SPR741 in combination with azithromycin (AZ), aztreonam (AZT), clarithromycin (CLR), fusidic acid (FA), meropenem (MEM), mupirocin (MUP), rifampicin (RIF), and retapamulin (RET) was assessed using a susceptibility testing method based on CLSI guidelines M7-A10. Assays were performed against 25 Escherichia coli (Ec), 25 Klebsiella pneumoniae (Kp), and 17 Acinetobacter baumannii (Ab) MDR and clinical isolates, in Mueller-Hinton broth-II (cation adjusted) containing 0, 2 or 8 μg/mL SPR741. The minimum inhibitory concentration (MIC) was defined as the lowest concentration of antibiotic required to inhibit visible bacterial growth. MIC values were determined for each antibiotic/SPR741 combination and maximum reductions in MIC of the antibiotic in the combination were determined.

Combination with SPR741 resulted in potentiation of test antibiotics against isolates of Ab, Ec, and Kp. For Ab, potentiation followed the order FA>RIF>CLR>RET>MEM>AZ>AZT>MUP, with 128 fold reductions in MIC relative to antibiotic alone (MIC reduced from >128 to ≤2 μg/mL in the combination). For Ec, potentiation followed the order RIF>MUP=FA>AZ=RET>AZT>MEM=CLR; maximum RIF MIC reduction of >2000 fold (MIC reduced from >128 to <0.125 μg/mL). For Kp, potentiation followed the order RET>RIF=AZ=CLR>MUP>MEM=FA>AZT; the MIC of RET was reduced 32 fold (MIC reduced from 128 to 4 μg/mL).

### Example 2. Assessment of SPR741 Efficacy in Combination with Certain Antibiotics in Checkerboard Assays

Efficacy was assessed in checkerboard assays. The minimum inhibitory concentration (MIC) of SPR741, antibiotics, and combinations thereof was defined as the lowest concentration that inhibited growth of Ec ATCC 25922, Ab NCTC 12156 and Kp ATCC 43816. Ec BW25113, ΔtolC and ΔacrA were used to assess the contribution of the multi drug efflux pump AcrAB-TolC to susceptibility to the combinations. Interactions were assessed by calculating fractional inhibitory concentration indices (FICI) for each combination in which the MIC differed from compounds in isolation. Interactions were defined as: FICI>4, antagonism; 0.5-4, no interaction; <0.5, synergy. The minimum bactericidal concentration of combinations in the presence of 5% surfactant (Survanta) was also determined.

Of 22 antibiotics tested, the MIC of 7—azithromycin, clarithromycin (CLR), fusidic acid (FA), mupirocin, retapamulin (RET) rifampicin (RIF), telithromycin—against Ec and Kp was reduced 32-8,000-fold in the presence of 8-16 μg/mL SPR741; against Ab, similar potentiation was achieved with CLR, FA, RET, and RIF. SPR741 was able to potentiate antibiotics that are substrates of AcrAB-TolC, effectively circumventing the pump's contribution to intrinsic antibiotic resistance. RIF or CLR in combination with SPR741 were bactericidal at concentrations similar to the MIC irrespective of the presence of Survanta.

1. A method of treating a bacterial infection in a subject comprising administering a therapeutically effective amount of a combination of SPR741 and an antibiotic selected from retapamulin, telithromycin, aztreonam, and combinations thereof to the subject.

2. The method of claim 1, wherein the bacterial infection is an E. coli infection, a Klebsiella pneumoniae infection, or an Acinetobacter baumannii infection.

3. The method of claim 1, wherein the antibiotic is retapamulin.

4. The method of claim 1, wherein the antibiotic is telithromycin.

5. The method of claim 1, wherein the antibiotic is aztreonam.

6. The method of claim 1, wherein the subject is a mammal.

7. The method of claim 6, wherein the subject is a human patient.

8. The method of claim 3, wherein the retapamulin is administered as a topical formulation containing SPR741 and less than 1 mg retapamulin per gram formulation.

9. The method of claim 4, wherein the telithromycin is administered orally and 10 mg to 300 mg telithromycin are administered daily.

10. The method of claim 5, wherein aztreonam is administered intravenously and less than 250 mg aztreonam are administered per 30 minute intravenous infusion.

11. A pharmaceutical composition comprising SPR741 and at least one antibiotic selected from retapamulin, telithromycin, and aztreonam.

12. The pharmaceutical composition of claim 11 additionally comprising a pharmaceutically acceptable carrier.

13. The pharmaceutical composition of claim 11, wherein the antibiotic is retapamulin, the composition is a topical composition and the composition contains less than 1 mg retapamulin per gram formulation.

14. The pharmaceutical composition of claim 11, wherein the antibiotic is telithromycin, the composition is an oral dosage form formulated for once daily administration and the dosage form contains 10 mg to 300 mg telithromycin.

15. The pharmaceutical composition of claim 11, wherein the antibiotic is aztreonam, the composition is an injectable or intravenous composition, and the composition contains less than 250 mg aztreonam per injection or infusion.

16. The method of claim 2, wherein the antibiotic is retapamulin.

17. The method of claim 2, wherein the antibiotic is telithromycin.

18. The method of claim 2, wherein the antibiotic is aztreonam.

19. The method of claim 2, wherein the subject is a mammal.
